(19) United States
(12) Patent Application Publication
(10) Pub. No.: US 2008/0183173 A1
(43) Pub. Date: Jul. 31, 2008

(54) Skull drill

(75) Inventor: Walter Jozat, Bad Bramstedt (DE)

Correspondence Address: HORST M. KASPER, 13 FOREST DRIVE, WARREN, NJ, 07059, US

(73) Assignee: SOERING GMBH

(21) Appl. No.: 11/986,623

(22) Filed: Nov. 21, 2007

(30) Foreign Application Priority Data
Nov. 24, 2006 (DE) 20 2006 017 876.7

(51) Int. Cl. A61B 17/58; A61B 17/32; A61H 1/00

(52) U.S. Cl. 606/80; 606/169; 601/2

(57) ABSTRACT

An ultrasound converter (5) is furnished in the gripping part of a hand piece. The hand piece is connected to an ultrasound generator (7) through a connection line (6). The sonotrode (1,1′, 1″) is introduced through a corresponding opening in the front end of the hand piece and is screwed together with the ultrasound converter (5). The sonotrode (1,1′, 1″) is formed such that the distal end transitions into a larger diameter, the drill head (2,2′,2″). This drill head is furnished with an axial bore hole such that the drill crown (3,3′, 3″) is formed in the kind of a tube drill or a hole saw. The shape of the cutting teeth furnished at the front side of the crown (3,3′, 3″) of the drill is different depending on the case of application. A stop sleeve (4) is slid over the sonotrode, wherein the stop sleeve (4) is attached in the grip part of the ultrasound converter (5) changeable in length with a fine thread.

### BACKGROUND

1. Field of the Invention

The present invention concerns a drill and a device for drilling holes in or, respectively, through bone tissue, in particular for drilling through the skull cover.

2. Brief Description of the Background of the Invention Including Prior Art

Various devices and drills have become known in the state-of-the-art, which in principle all work similar to a conventional spiral drill. For example a two-stage trepan is described in the German printed patent document DE 3890213 C2, wherein the hole to be drilled is drilled in two stages through the skull bone. The so-called main drill drills into the skull cover and at the point in time of the break through of the main drill through the skull cover it is effected based on the special construction of the tool that the drill crown remains standing at the obtained position. This results in a sudden and reliable decoupling such that the two drills momentarily remain standing. Thus the main drill cannot penetrate into the brain and the outer drill crown cannot cut any further into the skull cover.

All these drills, which cut the bone tissue by way of a rotary motion, require a rigid guidance toward the skull in order that a running away of the drill during placement onto the skull bone is avoided. In addition, there exists the danger with a spiral drill that the spiral drill pulls itself into the bone tissue and thus lays the danger present of too deep a penetration. This danger was described in the initially recited patent document and was avoided by a very expensive construction.

It is perceived to be disadvantageous that the bone tissue has to be cut completely in order to drill a hole of for example 6 to 8 mm diameter in a skull.

### SUMMARY OF THE INVENTION

### 1. Purposes of the Invention

It is an object of the present invention to furnish a device and a drill, which enables an entering or, respectively, penetration of a skull bone without a complete cutting of the bone.

### 2. Brief Description of the Invention

This object is obtained by having the drill formed in the kind of a hole saw as a sonotrode and that the drill is put into an ultrasound vibration by way of an ultrasound converter attached at the shaft of the drill.

It has proved to be advantageous for drilling of holes under low production of chips through a skull bone with a ring shaped drill formed as a sonotrode that the drill is furnished on the front side with a kind of toothing. After the placement of the tool onto the skull bone here the ultrasound generator is switched on or turned on. The drill now penetrates into the skull bone under slight pressure. The bone dust generated during dry drilling is blown out from the ring slot through the oscillations such that the sonotrode does not block with straight guidance. The additionally generated smoke is removed from the drilling position by an applied suction device.

The reduction of the heat generated during drilling and of the therewith associated smoke can be made possible by feeding in of water. However it is here disadvantageous that a foaming of the water and of the bone mud disposed in the ring slot occurs and that the operation field is not any longer clearly recognized. Tests have been performed with various frequencies. Here it became clear that a good result was obtained in the region from 20 to 70 kHz. The best result was obtained with a frequency from 45 to 60 kHz and in particular at 55 kHz.

The construction of the drill as a so-called hole saw is associated with the advantage that only a small part of the skull bone has to be cut into parts. It is here also advantageous that the outer shape of the drill is formed slightly conical in order to prevent a clamping during the drilling process. It is also conceivable while the drill is formed cylindrical to reduce the outer diameter of the drill immediately behind the toothing by a few tenths of a mm. An investigation of the oscillation behavior of the sonotrode finally determines the kind and shape of the reduction of diameter. The cut out skull bone segment during drilling is received from the inner space of the drill head. A bore hole preferably disposed parallel to the axis is furnished in the rear region of the drill head and this bore hole prevents that a compression pressure builds up during penetration of the bone disk. In addition after the conclusion of the drilling process, the bone segment can be ejected from the drill head through this bore hole with a thin rod.

In order to avoid that the dura mater encephali does not become injured during passage of the front side of the sonotrode, there is a so-called stop sleeve slid over the sonotrode in axial direction. This stop sleeve is adjustable in its longitudinal extension corresponding to the thickness of the skull bone. This stop sleeve is not connected to the ultrasound converter such that the stop face is not subjected to the sound energy. The outer diameter of the stop face is selected such that the view onto the front side of the sonotrode is not interfered with.

Another solution for drilling through only the skull bone but not through the dura mater encephali resides in monitoring the current pickup of the apparatus. The resistance collapses at the point in time of the break through the skull bone and the current use sinks automatically. At the point in time of the current drop, the voltage supply of the ultrasound generator can be interrupted with a corresponding electrical circuit.

Temperature measurements have indicated that the temperature can amount to up to 75 degrees centigrade depending on frequency and press on pressure. Such a temperature value does not need to damage the skull bone.

It is perceived to be particularly advantageous that the operator can operate the most different apparatuses with only a single ultrasound generator. For example for drilling the hole through the skull cover there is required only a corresponding hand piece with drill sonotrode, which hand piece is supplied by the ultrasound generator with energy through a feed line.

In case the ultrasound generator is equipped with an electronic hand piece recognition, then neither the surgeon nor the assistant have to be concerned with the setting of the required parameters, since the hand piece recognition calls the corresponding base values. Of course these base values can be still changed manually in case the surgeon deems this to be necessary.

The subject matter of the present invention is illustrated in more detail by way of drawings.

There is shown in:

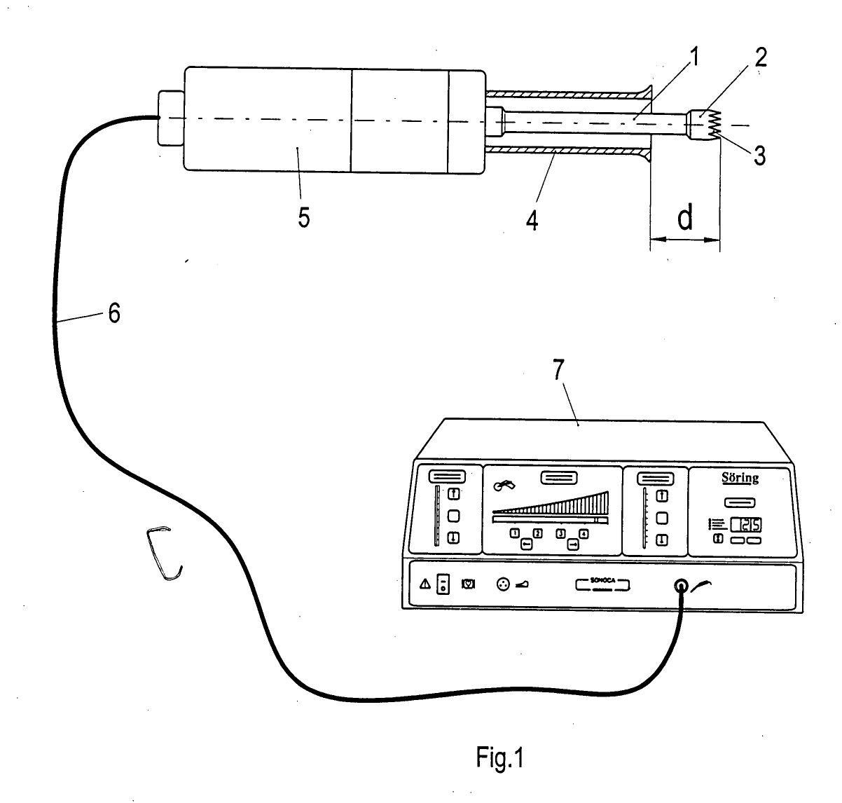

FIG. 1 a schematic arrangement of a drill sonotrode;

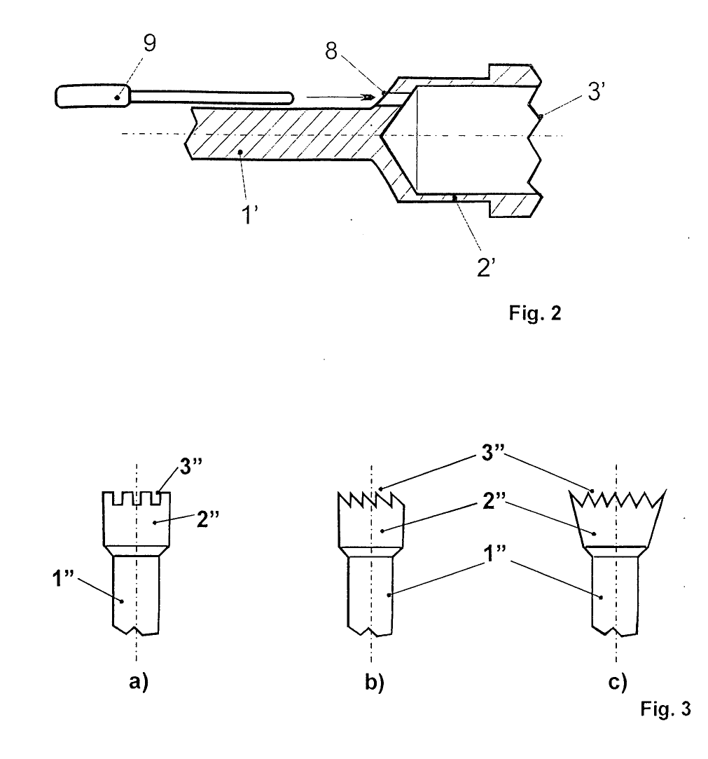

FIG. 2 a sectional view of a bore hole and of an ejection rod;

FIG. 3 different cut shapes a, b, and c of the crown of the drill.

A hand piece is shown in FIG. 1 in a simplified representation, where the ultrasound converter (5) is furnished in the gripping part of the hand piece. The hand piece is connected to an ultrasound generator (7) through the connection line (6). The sonotrode (1,1′, 1″) is introduced through a corresponding opening in the front end of the hand piece and is screwed together with the ultrasound converter (5). The sonotrode (1,1′, 1″) is formed such that the distal end transitions into a larger diameter, the drill head (2,2′,2″). This drill head is furnished with an axial bore hole such that the drill crown (3,3′, 3″) is formed in the kind of a tube drill or a hole saw. The shape of the cutting teeth furnished at the front side of the crown (3,3′, 3″) of the drill is different depending on the case of application.

A stop sleeve (4) is slid over the sonotrode, wherein the stop sleeve (4) is attached in the grip part of the ultrasound converter (5) changeable in length with a fine thread. This stop sleeve can exhibit a diameter enlargement at its free end in order that the stop face is large enough relative to the diameter of the drill head. The stop sleeve (4) can be axially displaced by way of the fine thread not illustrated here, depending on how thick the skull bone is in the region of the drilling position. This measure d is set by screwing in or, respectively, by screwing out of the stop sleeve (4) with an x-ray image. The stop sleeve (4) does not have any contact to the ultrasound converter and is thus free of oscillations. Such a stop sleeve is necessary in order for avoiding to injure the dura mater encepheli during the passage of the crown of the drill through the skull bone.

FIG. 2 shows an enlarged representation of the drill head (2,2′, 2″) of FIG. 1. It becomes clear from this figure that the shaft region of the drill head exhibits a smaller diameter relative to the crown region (3,3′, 3″). This situation is helpful as mentioned initially, since therewith a clamping of the drill head (2,2′, 2″) in the bore hole is prevented.

Three different forms of the drill crown (3,3′, 3″) are shown in FIG. 3 with the designation a, b, and c; in figure a the cutting teeth are formed rectangular, in figure b a clear saw tooth shape is shown, wherein it is here assumed that the drill head performs a small rotary motion. Figure c shows a drill crown with a toothing as known from FIGS. 1 and 2, however the outer diameter of the drill head (2,2′, 2″) is not cylindrical but instead slightly conically formed. This form again is to prevent a clamping of the drill head in the bore hole. Of course also other shapes of a drill crown are conceivable.

### LIST OF REFERENCE NUMERALS

1,1′, 1″ sonotrode

2,2′, 2″ drill head

3,3′, 3″ drill crown

4 stop sleeve

5 ultrasound converter

6 connection line

7 ultrasound generator

8 bore hole

9 ejection rod

d thickness of the skull bone

1. A drill for drilling bore holes in or, respectively, through bone tissue in particular for drilling through a skull cover, wherein the drill is formed as a sonotrode (1,1′, 1″), is formed like a tube in a distal region, and is formed with a toothing on a front side.

2. The drill for drilling of bore holes according to claim 1 wherein the sonotrode (1,1′, 1″) is excited to a frequency of from 20 to 70 kHz, preferably from 50 to 60 kHz.

3. The drill for drilling bore holes according to claim 1 wherein flanks of teeth of a drill crown (3,3′, 3″) are disposed under an angle of 45 degrees.

4. The drill for drilling bore holes according to claim 1 wherein flanks of teeth of a drill crown (3,3′, 3″) are inclined differently and exhibit a shape of a saw.

5. The drill for drilling bore holes according to claim 1 wherein the flanks of teeth of a drill crown (3,3′, 3″) run parallel to a center axis and leave a gap in each case between each other.

6. The drill for drilling bore holes according to claim 1 wherein an outer diameter of a drill crown (3,3′, 3″) is slightly larger than an outer diameter of a drill head (2,2′, 2″).

7. The drill for drilling bore holes according to claim 1 wherein an outer diameter of a drill head (2,2′, 2″) runs conically from a drill crown (3,3′, 3″) rearward.

8. The drill for drilling bore holes according to claim 1 wherein a bore hole (8) disposed in a rear region of a drill head (2,2′, 2″) and aligned parallel to an axis is furnished as a ventilation and ejection opening.

9. The drill for drilling bore holes according to claim 8 further comprising an ejection rod (9) as a kit part.

10. The drill for drilling bore holes according to claim 1 further comprising an axially adjustable stop sleeve (4) slid over the sonotrode (1,1′, 1″).

11. The drilling for drilling bore holes according to claim 10, wherein the stop sleeve (4) exhibits a dish like stop face on its front side.

12. The drill for drilling bore holes according to claim 10 wherein an axial adjustment is performed by way of a fine thread disposed on a surface of the stop sleeve (4).

13. The drill for drilling bore holes according to claim 10 wherein the axial adjustment is performed by a sliding seed, wherein the stop sleeve (4) is fixed with a clamping screw (not illustrated).

14. The drill for drilling bore holes according to claim 1 wherein a voltage supply of an ultrasound generator is interrupted after a penetration of a skull bone.

15. The drill for drilling bore holes according to claim 14, wherein an interruption of a voltage supply is triggered by a drop in the current use based on a lower resistance after a breaking through a skull bone.

16. A device for drilling bore holes comprising
an ultrasound generator (7);
a connection line (6);
an ultrasound converter (5) disposed in a hand piece;
a sonotrode (1,1′, 1″) formed as a drill and inserted into the ultrasound converter (5).

17. The device for drilling bore holes according to claim 16 wherein flanks of teeth of a drill crown (3,3′, 3″) are disposed under an angle of 45 degrees.

18. The device for drilling bore holes according to claim 16 wherein flanks of teeth of a drill crown (3,3′, 3″) are differently inclined and exhibit a shape of a saw.

19. The device for drilling bore holes according to claim 16 wherein flanks of teeth of a drill crown (3,3′, 3″) run parallel to a center axis and in each case leave a gap between each other.

20. The device for drilling bore holes according to claim 16 wherein an outer diameter of a drill crown (3,3′, 3″) is slightly larger than an outer diameter of a drill head (2,2′, 2″).

21. The device for drilling bore holes according to claim 16 wherein an outer diameter of a drill head (2,2′, 2″) runs conically from a drill crown (3,3′, 3″) rearward.

22. The device for drilling bore holes according to claim 16 further comprising a bore hole (8) disposed parallel to an axis and disposed in the rear region of a bore head (2,2′, 2″) and serving as a ventilation and ejection opening.

23. The device for drilling bore holes according to claim 22 further comprising a so-called ejection rod (9) furnished as a kit part.

24. The device for drilling of bore holes according to claim 16 wherein a stop sleeve (4) is attached in a hand piece casing.

25. The device for drilling bore holes according to claim 16 wherein a stop sleeve (4) is screwed into a hand piece casing with a fine thread.

26. The device for drilling bore holes according to claim 16 wherein an axial placing of a stop sleeve (4) is performed by a sliding seat, wherein the stop sleeve is fixed by a clamping screw (not illustrated here).

27. The device for drilling bore holes according to claim 16 wherein a voltage supply of an ultrasound generator is interrupted after a pushing through of a skull bone.

28. The device for drilling bore holes according to claim 16 wherein an interruption of a voltage supply is triggered by a sinking of a current use based on a smaller resistance after a breaking through a skull bone.
